(19) United States
(12) Patent Application Publication
(10) Pub. No.: US 2024/0023839 A1
(43) Pub. Date: Jan. 25, 2024

(54) BLOOD COMPONENT MONITORING DEVICE, NON-TRANSITORY COMPUTER-READABLE RECORDING MEDIUM, AND BLOOD COMPONENT MONITORING SYSTEM

(71) Applicant: TANITA CORPORATION, Tokyo (JP)

(72) Inventor: Satoshi KOIDE, Tokyo (JP)

(73) Assignee: TANITA CORPORATION, Tokyo (JP)

(21) Appl. No.: 18/478,385

(22) Filed: Sep. 29, 2023

Related U.S. Application Data
(63) Continuation of application No. PCT/JP2022/014841, filed on Mar. 28, 2022.

(30) Foreign Application Priority Data
Mar. 30, 2021 (JP) 2021-058540

(51) Int. Cl. A61B 5/145

(52) U.S. Cl. CPC A61B 5/14532; A61B 2560/0487

(57) ABSTRACT

A blood component monitoring device is a blood component monitoring device configured to monitor a blood component. The blood component monitoring device includes a concentration acquisition circuit configured to acquire the glucose correlation value that correlates with the blood glucose concentration and the neutral lipid correlation value that correlates with the blood neutral lipid concentration. The blood component monitoring device includes an information generation circuit configured to generate the information related to the blood status on the basis of at least one of the variation patterns of the glucose correlation value and the neutral lipid correlation value caused by a meal.

### TECHNICAL FIELD

The present invention relates to a blood component monitoring device, A non-transitory computer-readable recording medium, and a blood component monitoring system.

### BACKGROUND ART

WO2019/021358A1 discloses a dietary advice providing system.

This dietary advice providing system provides dietary advice on the basis of the blood sugar level measured before a meal, the blood sugar level measured after the meal, and the time the blood sugar level was obtained.

### SUMMARY OF INVENTION

However, such a dietary advice providing system provides the advice regarding diet on the basis of the blood sugar level only. Thus, although lipids are included in the blood components that vary due to the meal, the dietary advice providing system can only provide the dietary advice regarding the sugars.

The present invention has been conceived in light of the problems mentioned above, and an object thereof is to enable provision of multifaceted information on a blood status in consideration of not only sugars, but also other blood components that vary due to a meal.

According to an aspect of the present invention, a blood component monitoring device that monitors a blood component is provided. The blood component monitoring device includes the concentration acquisition circuit configured to acquire the glucose correlation value that correlates with the blood glucose concentration and the neutral lipid correlation value that correlates with the blood neutral lipid concentration. The blood component monitoring device includes the information generation circuit configured to generate the information related to the blood status based on at least one of the variation patterns of the glucose correlation value and the neutral lipid correlation value caused by a meal.

According to this aspect, the information related to the blood status is generated on the basis of the variation patterns of the glucose correlation value that correlates with the blood glucose concentration and the neutral lipid correlation value that correlates with the blood neutral lipid concentration.

Therefore, compared with a case in which the information is generated on the basis of the blood sugar level only, it is possible to provide multifaceted information related to the blood status, in which not only sugars but also other blood components that are varied by a meal are taken into consideration.

In addition, because the information related to the blood status is generated by using the variation patterns, it is possible to provide the information related to the blood status at appropriate timings corresponding to the variation.

### BRIEF DESCRIPTION OF DRAWINGS

FIG. 1 is a block diagram showing an example of a hardware configuration of a blood component monitoring device according to a first embodiment.

FIG. 2 is a functional block diagram showing an example of a functional configuration of the blood component monitoring device according to the first embodiment.

FIG. 3 is a flowchart showing an example of an operation of a blood component monitoring process according to the first embodiment.

FIG. 4 is a flowchart showing an example of an advice generation process according to the first embodiment.

FIG. 5 is a flowchart showing an example of a combination advice generation process according to the first embodiment.

FIG. 6 is a diagram showing variation patterns of a glucose correlation value according to the first embodiment.

FIG. 7 is a diagram showing variation patterns of a neutral lipid correlation value according to the first embodiment.

FIG. 8 is a diagram showing an example of a data table showing a relationship between combinations of variation patterns when a neutral lipid correlation value is normal and advices.

FIG. 9 is a diagram showing an example of a data table showing a relationship between the combinations of the variation patterns when postprandial hyperlipidemia is suspected and the advices.

FIG. 10 is a diagram showing an example of a data table showing a relationship between the combinations of the variation patterns when hyperlipidemia is suspected and the advices.

FIG. 11 is a diagram showing an example of a data table showing a relationship between the combinations of the variation patterns when hypolipidemia is suspected and the advices.

FIG. 12 is a flowchart showing an example of exceedance advice process according to the first embodiment.

FIG. 13 is a diagram showing an example of real-time advice.

### DESCRIPTION OF EMBODIMENTS

### First Embodiment

In the following, a first embodiment will be described with reference to the attached drawings.

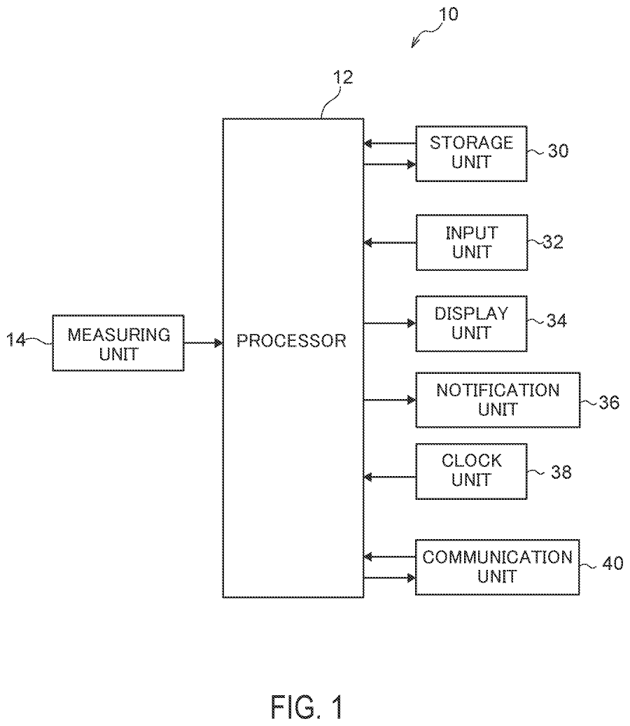

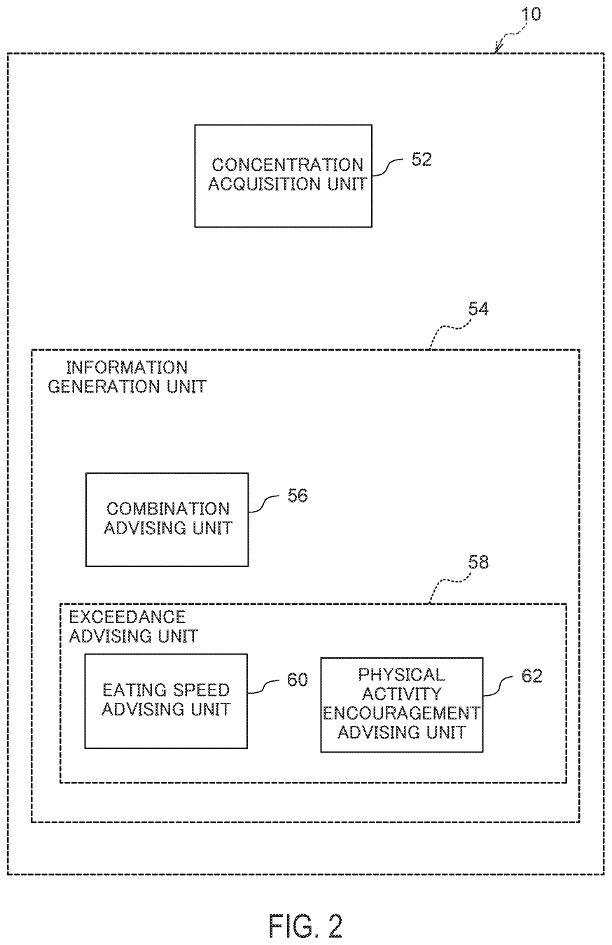

FIG. 1 is a block diagram showing an example of a hardware configuration of a blood component monitoring device 10 according to a first embodiment. FIG. 2 is a functional block diagram showing an example of a functional configuration of the blood component monitoring device 10 according to the first embodiment.

The blood component monitoring device 10 is a device that monitors the blood components of a user. The blood components to be monitored include glucose (blood glucose) and neutral lipids (neutral fats).

The blood component monitoring device 10 is, as an example, configured by a wearable device worn by the user. A wearing position of the blood component monitoring device 10 includes, for example, a wrist of the user.

(Hardware Configuration)

As shown in FIG. 1, the blood component monitoring device 10 is mainly formed of a processor 12 that forms a computer. The processor 12 is connected to a measuring unit 14, a storage unit 30, an input unit 32, a display unit 34, a notification unit 36, a clock unit 38, and a communication unit 40.

The measuring unit 14 is formed of a detection sensor that detects the blood components of the user. The detection sensor forming the measuring unit 14 detects a correlation value correlated with the concentration of blood components in the body noninvasively without harming the user.

A known detection method is used as a detection method of the correlation value that correlates with the concentration of the blood components. In this detection method, as an example, a near infrared ray is used. By using the near infrared ray, the measuring unit 14 continuously detects the correlation value that correlates with the concentration of the blood components in the body of the user.

The blood components to be detected by the measuring unit 14 are glucose and neutral lipids. The measuring unit 14 continuously detects the glucose correlation value that correlates with the blood glucose concentration and the neutral lipid correlation value that correlates with the blood neutral lipid concentration, and sends both detection signals to the processor 12. Thereby, the blood component monitoring device 10 continuously detects the glucose correlation value and the neutral lipid correlation value and acquires the temporal change of the glucose correlation value and the neutral lipid correlation value.

The glucose correlation value correlates with the blood glucose concentration, and the blood glucose concentration can be estimated from the glucose correlation value. The neutral lipid correlation value correlates with the blood neutral lipid concentration, and the blood neutral lipid concentration can be estimated from the neutral lipid correlation value.

Here, the concept indicated by the glucose correlation value includes the blood glucose concentration. In addition, the concept indicated by the neutral lipid correlation value includes the blood neutral lipid concentration.

In this embodiment, although a case in which the glucose correlation value that correlates with the blood glucose concentration and the neutral lipid correlation value that correlates with the blood neutral lipid concentration are detected will be described, this embodiment is not limited thereto. For example, in addition to the glucose correlation value and the neutral lipid correlation value, correlation values that correlate with blood concentrations of other blood components may also be detected.

In addition, in this embodiment, although a case in which the correlation value that correlates with the blood concentration is measured noninvasively will be described, this embodiment is not limited thereto. As an example, blood in the body may be collected to measure the correlation value that correlates with the blood concentration.

The storage unit 30 stores data so as to be readable by the processor 12. In the storage unit 30, a blood component monitoring program for controlling the operation of the blood component monitoring device 10 is stored.

The storage unit 30 functions as a storage medium for storing the blood component monitoring program that realizes a function of an information processing device of this embodiment. The storage unit 30 is formed of a non-volatile memory (ROM: Read Only Memory), a volatile memory (RAM: Random Access Memory), and so forth.

In addition, the storage unit 30 stores the data to be used by the blood component monitoring program so as to be readable.

For example, the storage unit 30 sequentially stores the glucose correlation values and the neutral lipid correlation values acquired by the measuring unit 14. In addition, in the storage unit 30, a voice data indicating the advice that is used in accordance with the measurement result of the measuring unit 14 is stored so as to be able to be played back as voice. Furthermore, in the storage unit 30, a character data indicating the advice that is used in accordance with the measurement result of the measuring unit 14 is stored so as to be able to be displayed as characters.

Specifically, the storage unit 30 stores a data table indicating a relationship between the advice and the variation patterns of the glucose correlation value and the neutral lipid correlation value caused by a meal. In addition, the storage unit 30 stores the advice that is to be used when at least one of the glucose correlation value and the neutral lipid correlation value exceeds a predetermined value.

The storage unit 30 stores advice about how to eat a meal that is to be used when the glucose correlation value exceeds a first threshold value. The advice about how to eat a meal includes advice about how to reduce a speed of eating a meal. The storage unit 30 may store the data table in which these advices are stored.

In addition, the storage unit 30 stores advice for encouraging physical activity that is to be used when the neutral lipid correlation value exceeds a second threshold value.

The input unit 32 sends the data input by the user to the processor 12. The input unit 32 functions as an input interface that receives an input operation performed by the user. As an example, the input unit 32 is formed of a plurality of operation buttons and numeric buttons.

The display unit 34 displays according to the data from the processor 12. The display unit 34 notifies the user of the measurement result, the advice, and so forth by means of a display. The device that notifies the user by means of a display includes a light emitting diode or a liquid crystal display such as a LCD (Liquid Crystal Display), etc., and the display unit 34 of this embodiment is formed of the liquid crystal display, as an example.

The notification unit 36 performs the notification according to the data from the processor 12. The notification unit 36 informs the user of a guidance, a warning sound, the advice, or the like by sound. The device that performs the notification with sound includes a piezoelectric buzzer, a speaker, or the like, and the notification unit 36 of this embodiment is formed of the speaker, as an example.

The clock unit 38 indicates the current year, month, and date, and measures time. The clock unit 38 outputs the current year, month, date, and time to the processor 12.

The communication unit 40 enables transmission and reception of the data between the processor 12 and an external device. The communication unit 40 forms an interface for transmitting and receiving the data. The communication unit 40 is formed of a hardware that performs the communication by using USB (universal serial bus), Bluetooth®, wireless LAN, short-range wireless communication (FeliCa®, etc.), LPWA, cellular phone lines such as 4G and 5G, or the like.

When the blood component monitoring program described above is supplied from the external device, the communication unit 40 receives the blood component monitoring program from the external device and sends it to the processor 12. The processor 12 stores the thus-received blood component monitoring program in the storage unit 30. When the communication unit 40 is formed of an Internet connection device, the communication unit 40 receives the blood component monitoring program from a server, etc., which is the external device, through a network such as an Internet network and a telephone network.

As an example, the processor 12 is formed of a central processing unit (CPU). The processor 12 reads a program stored in the storage unit 30 and operates in accordance with the read program. Thus, the processor 12 controls respective units of the blood component monitoring device 10 to perform a blood component monitoring method.

In addition, the processor 12 displays the advice, etc. on the display unit 34, notifies the advice, etc. via the notification unit 36, and transmits the advice, etc. to the external device via the communication unit 40.

(Functional Block)

As shown in FIG. 2, the blood component monitoring device 10 includes a concentration acquisition unit 52 and an information generation unit 54 serving as an information generation unit. The information generation unit 54 includes a combination advising unit 56 and an exceedance advising unit 58. The exceedance advising unit 58 includes an eating speed advising unit 60 that gives the advice regarding eating as an example and an physical activity encouragement advising unit 62.

The functions of the respective units of the blood component monitoring device 10 are realized by the processor 12 executing the blood component monitoring program that is a software program read from the storage unit 30.

At least one of the functions of the respective units of the blood component monitoring device 10 may be realized by an individual hardware such as ASIC, etc. In addition, the respective units of the blood component monitoring device 10 may also be realized by a combination of the software program and the individual hardware.

<Concentration Acquisition Unit>

The concentration acquisition unit 52 acquires the glucose correlation value that correlates with the blood glucose concentration and the neutral lipid correlation value that correlates with the blood neutral lipid concentration.

The concentration acquisition unit 52 estimates the concentrations of both blood components from an acquired value that is obtained by acquiring the correlation value that correlates with the concentration of the blood component of the user from the detection sensor of the measuring unit 14. The correlation value to be estimated includes the glucose correlation value that correlates with the blood glucose concentration and the neutral lipid correlation value that correlates with the blood neutral lipid concentration.

In a case in which the blood in the body is collected to acquire the correlation value that correlates with the blood concentration, the correlation values are the blood glucose concentration and the blood neutral lipid concentration.

The concentration acquisition unit 52 continuously acquires the glucose correlation value and the neutral lipid correlation value to acquire the temporal change of the glucose correlation value and the neutral lipid correlation value. The concentration acquisition unit 52 records the glucose correlation value and the neutral lipid correlation value, which are acquired continuously, in the storage unit 30 in a readable manner in association with the date and the time obtained from the clock unit 38.

<Information Generation Unit>

The information generation unit 54 generates the information related to the blood status on the basis of the variation patterns of the glucose correlation value and the neutral lipid correlation value caused by a meal. The information generated by the information generation unit 54 includes alert, advice, or the like. In this embodiment, the information generation unit 54 generates the advice.

The advice to be generated includes, as an example, the advice regarding health. The advice regarding health at least includes the advice regarding eating or the advice regarding the physical activity.

The advice regarding eating includes the advices regarding an amount of meal, a content of meal, a mealtime duration, an order of eating meal, and a cooking method of meal.

The variation pattern of the glucose correlation value and the neutral lipid correlation value caused by a meal indicates, when the user eats a meal, a change in the glucose correlation value and the neutral lipid correlation value that appear between before and after a meal.

Whether or not the user has started a meal is determined on the basis of the variation pattern of the glucose correlation value and the neutral lipid correlation value stored in the storage unit 30 or on the basis of an input state of the operation buttons of the input unit 32. In this embodiment, as an example, it is determined that a meal is started when the operation buttons of the input unit 32 are turned on.

Neutral lipids serve as a source of energy. However, if neutral lipids are taken excessively, they are taken up by the body as fat. Therefore, by measuring variation in a neutral lipid level in addition to that in a glucose level, it is possible to check excessive intake of glucose and lipids. By doing so, it is made possible to notify the need for improvement in the meal content and for the physical activity.

The relationship between fat and diabetes is known to be related to arteriosclerosis. Excessive glucose intake is known to increase a tendency to store neutral fat. Consumption of sweet foods when the concentration of neutral lipids is high is known to increase a risk of obesity. Therefore, simultaneous measurement of the neutral lipid level and the glucose level is helpful in preventing these problems.

Here, the basic advice required for a combination of glucose and neutral lipids will be described.

“Case with High Glucose Level and High Neutral Lipid Level”

The advice for a case with a high glucose level and a high neutral lipid level includes the following.

An example of the advice regarding the amount of meal includes “Please refrain from eating and drinking.”, etc. An example of the advice regarding the meal content includes “Sugar-free tea is recommended.”, etc. Examples of the advice regarding the mealtime duration include “Eat food slowly.”, “Chew food more thoroughly.”, and so forth.

An example of the advice regarding the order of eating meal includes “Eat a meal in this order: soup, vegetables, main dish, and rice.”, etc. An example of the advice regarding the cooking method includes “Change rice to five-grain rice or fried rice.”, etc.

Examples of the advice regarding the physical activity includes “Participate in physical activity.”, “Take a 30-minute walk.”, “Cycle for 30 minutes.”, “Get off one stop early and walk back home.”, and so forth.

“Case with Low Glucose and High Neutral Lipids”

An example of the advice for a case with a low glucose level and a high neutral lipid level includes the following.

First, advice to lower the neutral lipid level is given. In addition, it is desirable to give advice to lower sugar (glucose) level because of the sensitive nature of neutral lipids to sugars (glucose).

An example of the advice regarding the amount of meal includes “Try to avoid foods high in sugar and especially fatty foods.”, etc.

Examples of the advice regarding the meal content include “Avoid foods containing fats (fried foods).”, “Avoid fried foods such as fries, and eat boiled foods, steamed foods, and so forth.”, “Grains are acceptable to eat.”, and so forth. Furthermore, as the advice regarding the meal content, it is further preferred to give the advice to lower sugars such as “It is advisable to eat sugar-free snacks, foods and beverages that do not contain carbohydrates.”, “It is advisable to eat sugar-free tea, low-calorie desserts and snacks, and desserts that do not contain carbohydrates.”, and so forth.

As described above, the advice to lower the neutral lipid level may be given preferentially, and the advice to lower the sugar (glucose) level may further be given.

Examples of the advice regarding the physical activity include “Participate in physical activity.”, “Take a 30-minute walk.”, “Cycle for 30 minutes”, “Get off one stop early and walk back home.”, and so forth.

“Case with a High Glucose Level and a Low Neutral Lipid Level”

An example of the advice for a case with a high glucose level and a low neutral lipid level includes the following. In this case, there is no restriction for lipids.

In a case with a high glucose level and a low neutral lipid level, the advice is given to lower the glucose level only.

Examples of the advice regarding the amount of meal and the meal content include “Avoid carbohydrates (grains) and sweet foods.”, “Eat sugar-free tea, and sugar-free desserts that do not contain carbohydrates.” and so forth. Examples of the advice regarding the mealtime duration include “Eat food slowly.”, “Chew food more thoroughly.”, and so forth.

An example of the advice regarding the order of eating meal includes “Eat meal in this order: soup, vegetables, main dish, and rice.”, etc.

The advice regarding the cooking method recommends the use of oil to reduce the absorption of sugars. Examples of the advice regarding the cooking method include “Change rice to five-grain rice or fried rice.”, “Include side dishes prepared by using oil.” and so forth. As described above, in a case with a high glucose level and a low neutral lipid level, even if the advice increases the blood neutral lipid concentration slightly, it is possible to give the advice to lower the blood glucose concentration with priority.

“Case with a Low Glucose Level and a Low Neutral Lipid Level”

In a case with a low glucose level and a low neutral lipid level, it is notified that everything is in a normal state by a message such as “You are in normal state.”.

In a case in which the glucose level and the neutral lipid level are too low, the advice to encourage the user to take a meal is given. In addition, when it is outside the mealtime period, the advice to encourage the user to take a snack is given.

In addition, a fundamental concept behind the variation pattern of the glucose level or the neutral lipid level and the corresponding advice will be described.

In a case in which the glucose level (the blood glucose value) is increased rapidly, the advice on how to eat is given. As examples of the advice on how to eat, “Eat food slowly.”, “Chew food more thoroughly.” and so forth are included.

Examples of the advice regarding the cooking method include “Change rice to fried rice.”, “Change udon noodles to yakisoba noodles.”, and so forth. An example the advice regarding the order of eating meal includes “Eat a meal in this order: vegetables, main dish (meat/fish), and rice.”, etc., and an example of the advice regarding on food combination includes “Eat fried foods.”, etc.

In a case in which the neutral lipid level is not reduced even when a certain time period has been elapsed since the start of a meal, the advice regarding the physical activity is given. An example of the advice regarding the physical activity includes “Participate in aerobic activity.”, etc. The aerobic activity includes, as an example, “30-minute walk” or “15-minute cycling”, and so forth.

In addition, if the neutral lipid level is high in the fasting state, the advice is given to review the meal content, to encourage reflection on overeating or excessive drinking, or on the future meal content. In a case in which the glucose (sugar) level is low in the fasting state and the user is in a hypoglycemic state, the advice is given to encourage the user to take sugar with candy, etc.

When there is a blood glucose value spike in which the glucose level is rapidly increased, the advice includes “Eating speed is too fast, chew food more slowly and thoroughly.” and “Chew each mouthful 20 times.”. An example of the advice regarding the order of eating meal includes “Eat meal in this order: vegetables, side dishes, and rice.”, etc. Examples of the advice regarding the cooking method include “Eat foods prepared by using oil.”, “Eat carbohydrates prepared by using oil.”, and so forth.

In a case in which appearance of the maximum value of a postprandial neutral lipid level is slow and the glucose level (the blood concentration) is not reduced, examples of the advice include “Eat more vegetables.”, “Eat a meal in this order: vegetables, side dishes, and rice.”, “Avoid eating foods prepared by using oil.”, “Eat main dish prepared without using oil, such as steamed foods and boiled foods.”, “Avoid eating meat, and change it with fish.” and so forth.

The information generation unit 54 generates advice on the basis of the basic advice corresponding to the combination of the glucose level and the neutral lipid level described above and the basic advice corresponding to the variation pattern of the glucose level or the neutral lipid level.

[Combination Advising Unit]

The combination advising unit 56 generates advice on the basis of the combinations of the variation patterns of the glucose correlation value and the neutral lipid correlation value.

The combination advising unit 56 classifies the variation patterns of the glucose correlation value into four patterns as an example.

The four patterns of the glucose correlation value include a glucose normal pattern, in which the variation pattern of the glucose correlation value is close to the normal value, and a postprandial hyperglycemia pattern, in which the glucose correlation value is increased rapidly temporarily after a meal. In addition, the four patterns of the glucose correlation value include a diabetes pattern, in which the glucose correlation value is continued to be increased after a predetermined time after a meal, and a hypoglycemia pattern, in which the glucose correlation value is equal to or lower than the predetermined value and undergoes little change between before and after a meal.

In addition, the combination advising unit 56 classifies the variation patterns of the neutral lipid correlation value into the four patterns as an example.

The four patterns of the neutral lipid correlation value include a neutral-lipids normal pattern, in which the variation pattern of the neutral lipid correlation value is close to the normal value, and a postprandial hyperlipidemia pattern, in which the neutral lipid correlation value is continued to be increased after a predetermined time after a meal. In addition, the four patterns of the neutral lipid correlation value include a hyperlipidemia pattern, in which the neutral lipid correlation value is higher than the predetermined value between before and after a meal, and a hypolipidemia pattern, in which the neutral lipid correlation value is equal to or lower than the predetermined value and undergoes little change between before and after a meal.

The combination advising unit 56 generates advice in accordance with a combination of the four patterns of the glucose correlation value and the four patterns of the neutral lipid correlation value. In addition, the combination advising unit 56 notifies the generated advice via the display unit 34 and the notification unit 36.

The combination advising unit 56 may transmit the generated advice to other devices via the communication unit 40.

[Exceedance Advising Unit]

The exceedance advising unit 58 generates advice when at least one of the glucose correlation value and the neutral lipid correlation value exceeds the predetermined value.

The exceedance advising unit 58 generates advice on matters related to a meal, as an example, when the glucose correlation value exceeds the predetermined value. In addition, the exceedance advising unit 58 generates the advice regarding the physical activity, as an example, when the neutral lipid correlation value exceeds the predetermined value.

<Eating Speed Advising Unit>

The eating speed advising unit 60 generates advice to reduce the speed of eating a meal when the glucose correlation value exceeds the first threshold value.

As an example, the eating speed advising unit 60 generates the advice to reduce the speed of eating a meal at the time point when the sequentially measured glucose correlation value exceeds the first threshold value. The eating speed advising unit 60 notifies the generated advice via the display unit 34 and the notification unit 36.

The eating speed advising unit 60 may transmit the generated advice to other devices via the communication unit 40.

<Physical Activity Encouragement Advising Unit>

The physical activity encouragement advising unit 62 generates advice to encourage the user to participate in the physical activity when the neutral lipid correlation value exceeds the second threshold value.

As an example, the physical activity encouragement advising unit 62 generates the advice to encourage the user to participate in the physical activity at the time point when the sequentially measured neutral lipid correlation value exceeds the second threshold value. The physical activity encouragement advising unit 62 notifies the generated advice via the display unit 34 and the notification unit 36.

The physical activity encouragement advising unit 62 may transmit the generated advice to other devices via the communication unit 40.

(Description of Operation)

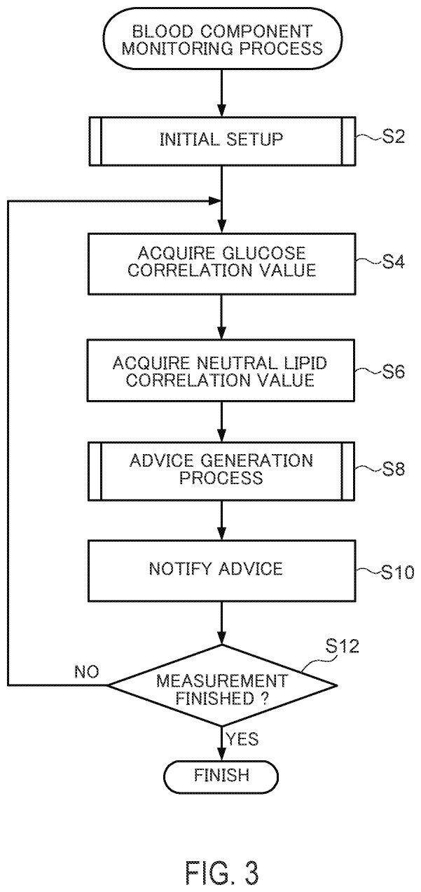

Next, the operation of the blood component monitoring device 10 will be described with reference to FIGS. 3 to 13 in accordance with processing procedures executed by the processor 12 of the blood component monitoring device 10.

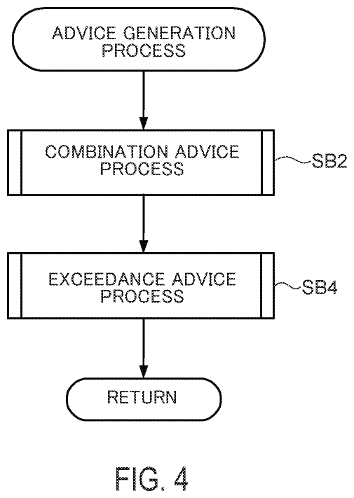

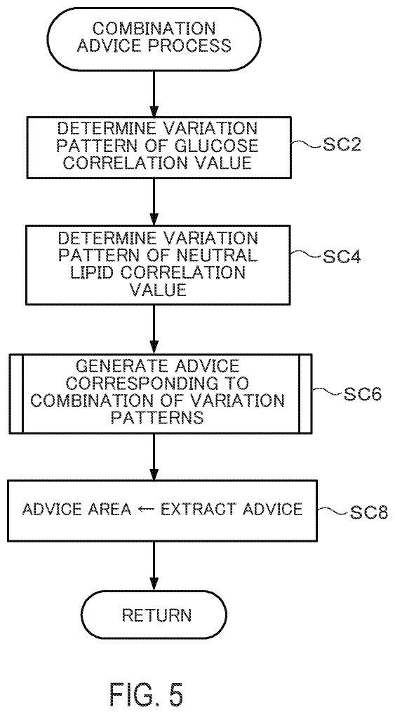

FIG. 3 is a flowchart showing an example of an operation of a blood component monitoring process according to the first embodiment. FIG. 4 is a flowchart showing an example of an advice generation process according to the first embodiment. FIG. 5 is a flowchart showing an example of a combination advice generation process according to the first embodiment.

When the processor 12 of the blood component monitoring device 10 executes the blood component monitoring process stored in the storage unit 30, the processor 12 performs initial setup (Step S2). In the initial setup, the processor 12 allocates, as examples, a data storage area in the storage unit 30 and an area for temporarily storing the data of the generated advice.

The processor 12 then obtains the glucose correlation value that correlates with the blood glucose concentration from the measuring unit 14 and records the obtained glucose correlation value in the storage unit 30 in association with the date and time obtained from the clock unit 38 (Step S4). In addition, the processor 12 obtains the neutral lipid correlation value that correlates with the blood neutral lipid concentration from the measuring unit 14 and records the obtained neutral lipid correlation value in the storage unit 30 in association with the date and time obtained from the clock unit 38 (Step S6). The processor 12 then executes the advice generation process (Step S8).

In the advice generation process, as shown in FIG. 4, the processor 12 executes a combination advice process (Step SB2).

In the combination advice process, as shown in FIG. 5, the processor 12 determines which pattern the variation pattern of the glucose correlation value belongs to on the basis of the temporal change of the glucose correlation value stored in the storage unit 30 (Step SC2).

In a case in which the stored amount of the glucose correlation value is insufficient and the glucose correlation value cannot be obtained for up to two hours after the start of a meal, the determination of the variation pattern of the glucose correlation value is not performed, and the process proceeds to Step SC4.

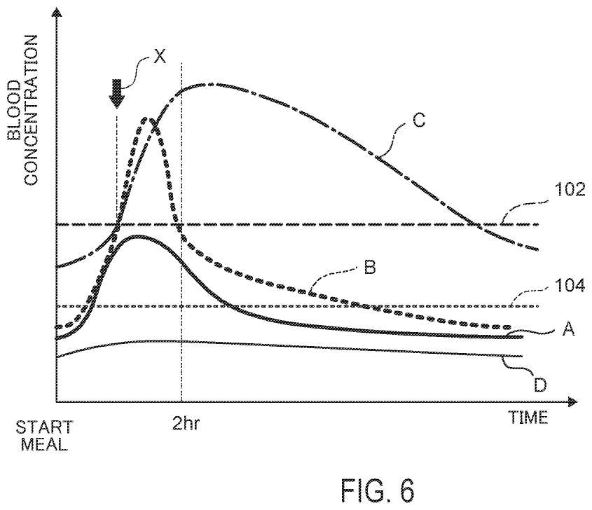

A specific method of determining the variation pattern will be described with reference to FIG. 6. FIG. 6 is a diagram showing the variation patterns of the glucose correlation value according to the first embodiment.

The variation pattern of the glucose correlation value is classified into a glucose normal pattern A, a postprandial hyperglycemia pattern B, a diabetes pattern C, and a hypoglycemia pattern D.

The glucose normal pattern A indicates the pattern in which the variation pattern of the glucose correlation value is close to the normal value. Specifically, the glucose normal pattern A indicates the pattern in which the blood glucose concentration indicated by the glucose correlation value is increased within two hours after the start of a meal, but it does not exceed a first predetermined value 102.

As an example, the first predetermined value 102 is defined as a glucose value so as to fall within a range from 160 mg/dL to 180 mg/dL. In this embodiment, as an example, the first predetermined value 102 is 180 mg/dL as the glucose value.

The postprandial hyperglycemia pattern B indicates the pattern in which the blood glucose concentration indicated by the glucose correlation value is increased rapidly temporarily after a meal. Specifically, the postprandial hyperglycemia pattern B indicates the pattern in which the blood glucose concentration indicated by the glucose correlation value is increased to exceed the first predetermined value 102 after the start of a meal, and subsequently, it is reduced so as to be equal to or lower than the first predetermined value 102 within two hours since the start of the meal.

As an example, the first predetermined value 102 is defined as the glucose value so as to fall within a range from 160 mg/dL to 180 mg/dL. In this embodiment, as an example, the first predetermined value 102 is 180 mg/dL as the glucose value.

The diabetes pattern C indicates the pattern in which the blood glucose concentration indicated by the glucose correlation value is continued to be increased after a predetermined time after a meal. Specifically, the diabetes pattern C indicates the pattern in which the blood glucose concentration indicated by the glucose correlation value is increased to exceed the first predetermined value 102 after the start of a meal, and it remains higher than the first predetermined value 102 even after two hours have passed since the start of the meal.

As an example, the first predetermined value 102 is defined as the glucose value so as to fall within a range from 160 mg/dL to 180 mg/dL. In this embodiment, as an example, the first predetermined value 102 is 180 mg/dL as the glucose value.

In this embodiment, although the first predetermined value 102 is used for the determination of the glucose normal pattern A, the determination of the postprandial hyperglycemia pattern B, and the determination of the diabetes pattern C, the present invention is not limited thereto. The predetermined value to be used for the determination of the glucose normal pattern A, the determination of the postprandial hyperglycemia pattern B, and the determination of the diabetes pattern C may be different values.

The hypoglycemia pattern D indicates the pattern in which the glucose correlation value is equal to or lower than the predetermined value and undergoes little change between before and after a meal. Specifically, the hypoglycemia pattern D indicates the pattern in which the blood glucose concentration indicated by the glucose correlation value is equal to or lower than a second predetermined value 104 before and after a meal. As an example, the second predetermined value 104 is 100 mg/dL as the glucose value.

In Step SC2, the processor 12 determines which of the glucose normal pattern A, the postprandial hyperglycemia pattern B, the diabetes pattern C, or the hypoglycemia pattern D, the temporal change of the glucose correlation value stored in the storage unit 30 belongs to.

Here, in this embodiment, although the blood glucose concentration indicated by the glucose correlation value is compared with each of the predetermined values 102 and 104 to classify each pattern, the present invention is not limited thereto. For example, it is possible to determine a suitable pattern for the user on the basis of a relative change between the past variation pattern stored in the storage unit 30 and the current variation pattern.

The processor 12 then determines which pattern the variation pattern of the neutral lipid correlation value belongs to on the basis of the temporal change of the neutral lipid correlation value stored in the storage unit 30 (Step SC4).

In a case in which the stored amount of the neutral lipid correlation value is insufficient and the neutral lipid correlation value cannot be obtained for up to two hours after the start of a meal, the determination of the variation pattern of the neutral lipid correlation value is not performed, and the process proceeds to Step SC6.

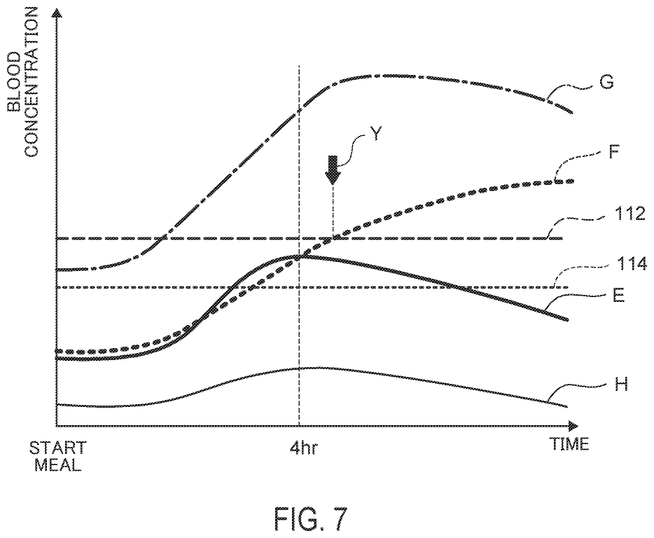

A specific method of determining the variation pattern will be described with reference to FIG. 7. FIG. 7 is a diagram showing the variation patterns of the neutral lipid correlation value according to the first embodiment.

The variation pattern of the neutral lipid correlation value is classified into a neutral-lipids normal pattern E, a postprandial hyperlipidemia pattern F, a hyperlipidemia pattern G, and a hypolipidemia pattern H.

The neutral-lipids normal pattern E indicates the pattern in which the variation pattern of the neutral lipid correlation value is close to the normal value. Specifically, the neutral-lipids normal pattern E indicates the pattern in which the blood neutral lipid concentration indicated by the neutral lipid correlation value is increased after the start of a meal and begins to fall by four hours after the start of the meal. In addition, in the neutral-lipids normal pattern E, the blood neutral lipid concentration indicated by the neutral lipid correlation value does not exceed a third predetermined value 112.

As an example, the third predetermined value 112 is defined as a neutral lipids value so as to fall within a range from 200 mg/dL to 250 mg/dL. In this embodiment, as an example, the third predetermined value 112 is 250 mg/dL as the neutral lipids value.

The postprandial hyperlipidemia pattern F indicates the pattern in which the neutral lipid correlation value is continued to be increased even if a predetermined time has passed since the start of a meal. Specifically, the postprandial hyperlipidemia pattern F indicates the pattern in which the blood neutral lipid concentration indicated by the neutral lipid correlation value is continued to be increased since the start of a meal and it exceeds the third predetermined value 112 after four hours since the start of the meal.

As an example, the third predetermined value 112 is defined as the neutral lipids value so as to fall within a range from 200 mg/dL to 250 mg/dL. In this embodiment, as an example, the third predetermined value 112 is 250 mg/dL as the neutral lipids value.

In this embodiment, although the third predetermined value 112 is used for the determination of the neutral-lipids normal pattern E and the determination of the postprandial hyperlipidemia pattern F, the present invention is not limited thereto. The predetermined value to be used for the determination of the neutral-lipids normal pattern E and the determination of the postprandial hyperlipidemia pattern F may be different values.

The hyperlipidemia pattern G indicates the pattern in which the neutral lipid correlation value exceeds the predetermined value between before and after a meal. Specifically, the hyperlipidemia pattern G indicates the pattern in which the blood neutral lipid concentration indicated by the neutral lipid correlation value exceeds a fourth predetermined value 114 between before and after a meal.

In this embodiment, as an example, the fourth predetermined value 114 is 150 mg/dL as the neutral lipids value.

Here, the normal value of the neutral lipids value is between 30 mg/dL and 149 mg/dL in the fasting state. High neutral lipids value is said to cause dyslipidemia or arteriosclerosis. In addition, if the neutral lipid level is low, it may be caused by diet, excessive physical activity, disease (hyperthyroidism, liver dysfunction), or constitution.

The hypolipidemia pattern H indicates the pattern in which the neutral lipid correlation value is equal to or lower than the predetermined value and undergoes little change between before and after a meal. Specifically, the hypolipidemia pattern H indicates the pattern in which the blood neutral lipid concentration indicated by the neutral lipid correlation value is equal to or lower than the fourth predetermined value 114 before and after a meal.

In this embodiment, as an example, the fourth predetermined value 114 is 150 mg/dL as the neutral lipids value.

In this embodiment, although the fourth predetermined value 114 is used for the determination of the hyperlipidemia pattern G and the determination of the hypolipidemia pattern H, the present invention is not limited thereto. The predetermined value to be used for the determination of the hyperlipidemia pattern G and the determination of the hypolipidemia pattern H may be different values.

In Step SC4, the processor 12 determines which of the neutral-lipids normal pattern E, the postprandial hyperlipidemia pattern F, the hyperlipidemia pattern G, or the hypolipidemia pattern H, the temporal change of the neutral lipid correlation value stored in the storage unit 30 belongs to.

Here, in this embodiment, although the blood neutral lipid concentration indicated by the neutral lipid correlation value is compared with each of the predetermined values 112 and 114 to classify each pattern, the present invention is not limited thereto. For example, it is possible to determine a suitable pattern for the user on the basis of a relative change between the past variation pattern stored in the storage unit 30 and the current variation pattern.

The processor 12 then generates advice on the basis of the combination of the determined variation pattern of the glucose correlation value and the determined variation pattern of the neutral lipid correlation value (Step SC6).

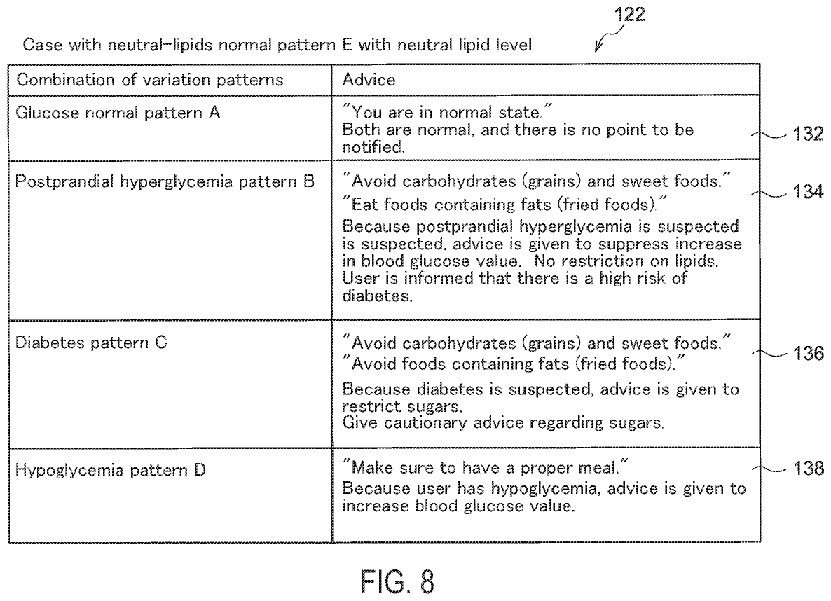

In this process of generating the advice, the data table stored in the storage unit 30 is used. The process of generating the advice will be described by using a first data table 122, a second data table 124, a third data table 126, and a fourth data table 128 shown in FIGS. 8 to 11.

FIG. 8 is a diagram showing an example of the first data table 122 showing a relationship between the combinations of the variation patterns when the neutral lipid correlation value is normal and the advices. The first data table 122 stores the advices that correspond to the combinations of the neutral-lipids normal pattern E, in which the variation pattern of the neutral lipid correlation value is close to the normal value, with each of the patterns A, B, C, and D indicating the variation patterns of the glucose correlation value.

When the variation pattern of the neutral lipid correlation value is the neutral-lipids normal pattern E and the variation pattern of the glucose correlation value is the glucose normal pattern A, the processor 12 extracts the voice data and the character data indicating advice 132 from the first data table 122.

With this combination of the patterns, the neutral lipid correlation value and the glucose correlation value are both normal, and there is no point to be notified.

As an example, the advice 132 is given as “You are in normal state.”.

When the variation pattern of the neutral lipid correlation value is the neutral-lipids normal pattern E and the variation pattern of the glucose correlation value is the postprandial hyperglycemia pattern B, the processor 12 extracts the voice data and the character data indicating advice 134 from the first data table 122.

Because postprandial hyperglycemia is suspected with this combination of the patterns, the advice is given to suppress the increase in the blood glucose value. In addition, although there is no restriction for lipids, because there is a high risk of diabetes, the user is informed accordingly. Here, lipids have the effect of suppressing the increase in the glucose level.

As examples, the advice 134 is given as “Avoid carbohydrates (grains) and sweet foods.” and “Eat foods containing fats (fried foods).”.

When the variation pattern of the neutral lipid correlation value is the neutral-lipids normal pattern E and the variation pattern of the glucose correlation value is the diabetes pattern C, the processor 12 extracts the voice data and the character data indicating advice 136 from the first data table 122.

Because diabetes is suspected with this combination of the patterns, the advice is given to restrict sugars. In addition, cautionary advice regarding lipids is given.

As examples, the advice 136 is given as “Avoid carbohydrates (grains) and sweet foods.” and “Avoid foods containing fats (fried foods).”.

When the variation pattern of the neutral lipid correlation value is the neutral-lipids normal pattern E and the variation pattern of the glucose correlation value is the hypoglycemia pattern D, the processor 12 extracts the voice data and the character data indicating advice 138 from the first data table 122.

Because this combination of the patterns indicates hypoglycemia, the advice is given to increase the blood glucose value.

As an example, the advice 138 is given as “Make sure to have a proper meal.”.

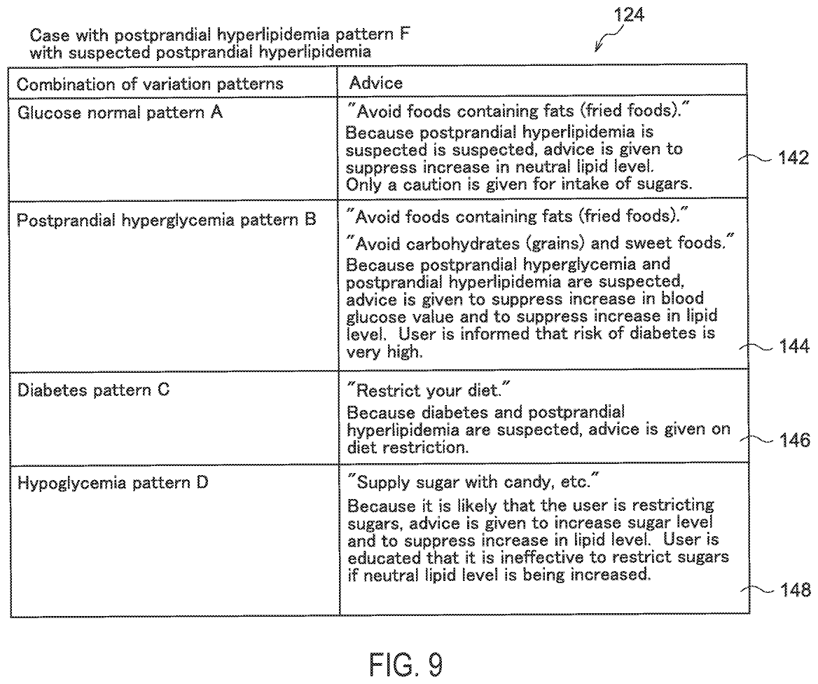

FIG. 9 is a diagram showing an example of the second data table 124 showing a relationship between the combinations of the variation patterns when postprandial hyperlipidemia is suspected and the advices.

The second data table 124 stores the advices that correspond to the combinations of the postprandial hyperlipidemia pattern F, in which the variation pattern of the neutral lipid correlation value indicates suspected postprandial hyperlipidemia, with each of the patterns A, B, C, and D indicating the variation patterns of the glucose correlation value.

When the variation pattern of the neutral lipid correlation value is the postprandial hyperlipidemia pattern F and the variation pattern of the glucose correlation value is the glucose normal pattern A, the processor 12 extracts the voice data and the character data indicating advice 142 from the second data table 124.

Because postprandial hyperlipidemia is suspected with this combination of the patterns, the advice is given to suppress the increase in the neutral lipid level. In addition, only a caution is given for intake of sugars.

As an example, the advice 142 is given as “Avoid foods containing fats (fried foods).”.

When the variation pattern of the neutral lipid correlation value is the postprandial hyperlipidemia pattern F and the variation pattern of the glucose correlation value is the postprandial hyperglycemia pattern B, the processor 12 extracts the voice data and the character data indicating advice 144 from the second data table 124.

Because postprandial hyperglycemia and postprandial hyperlipidemia is suspected with this combination of the patterns, the advice is given to suppress the increase in the blood glucose value and to suppress the increase in lipid level. In addition, because there is a very high risk of diabetes, the user is informed accordingly.

As examples, the advice 144 is given as “Avoid foods containing fats (fried foods).” and “Avoid carbohydrates (grains) and sweet foods.”.

When the variation pattern of the neutral lipid correlation value is the postprandial hyperlipidemia pattern F and the variation pattern of the glucose correlation value is the diabetes pattern C, the processor 12 extracts the voice data and the character data indicating advice 146 from the second data table 124.

Because diabetes and postprandial hyperlipidemia is suspected with this combination of the patterns, the advice is given to restrict diet.

As an example, the advice 146 is given as “Restrict your diet.”.

When the variation pattern of the neutral lipid correlation value is the postprandial hyperlipidemia pattern F and the variation pattern of the glucose correlation value is the hypoglycemia pattern D, the processor 12 extracts the voice data and the character data indicating advice 148 from the second data table 124.

Because it is likely that the user is restricting sugars with this combination of the patterns, the advice is given to increase sugars and to suppress the increase in lipid level. In addition, the advice is given to inform the user that there is no point in restricting sugars if the neutral lipid level is increased.

As an example, the advice 148 is given as “Supply sugar with candy, etc.”.

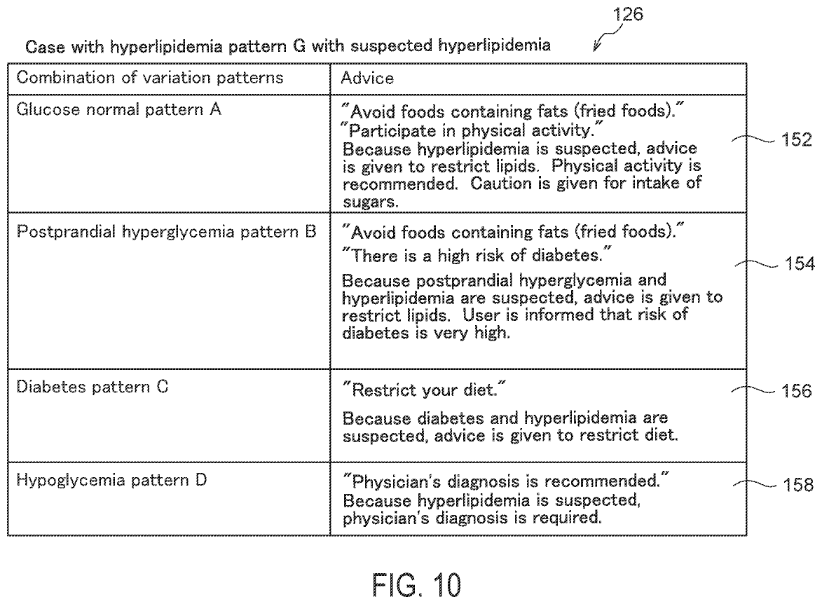

FIG. 10 is a diagram showing an example of the third data table 126 showing a relationship between the combinations of the variation patterns when hyperlipidemia is suspected and the advices.

The third data table 126 stores the advices that correspond to the combinations of the hyperlipidemia pattern G, in which the variation pattern of the neutral lipid correlation value indicates suspected hyperlipidemia, with each of the patterns A, B, C, and D indicating the variation patterns of the glucose correlation value.

When the variation pattern of the neutral lipid correlation value is the hyperlipidemia pattern G and the variation pattern of the glucose correlation value is the glucose normal pattern A, the processor 12 extracts the voice data and the character data indicating advice 152 from the third data table 126.

Because hyperlipidemia is suspected with this combination of the patterns, the advice is given to restrict lipids. In addition, the advice is given to encourage the user to participate in the physical activity. Furthermore, the advice is given to be careful for intake of sugars.

As examples, the advice 152 is given as “Avoid foods containing fats (fried foods).” and “Participate in physical activity.”.

Here, causes of the increased neutral lipid level include excessive intake of lipids and sugars or lack of physical activity. As a method to improve the neutral lipids level, the aerobic activity (such as walking, swimming, cycling, slow jogging, and so forth) is known, and it is desirable for the user to engage in the physical activity with an intensity of at least 3 Mets for a continuous duration of at least 30 minutes.

When the variation pattern of the neutral lipid correlation value is the hyperlipidemia pattern G and the variation pattern of the glucose correlation value is the postprandial hyperglycemia pattern B, the processor 12 extracts the voice data and the character data indicating advice 154 from the third data table 126.

Because postprandial hyperglycemia and hyperlipidemia are suspected with this combination of the patterns, the advice is given to restrict lipids. In addition, because there is a very high risk of diabetes, the user is informed accordingly.

As examples, the advice 154 is given as “Avoid foods containing fats (fried foods).” and “There is a high risk of diabetes.”.

When the variation pattern of the neutral lipid correlation value is the hyperlipidemia pattern G and the variation pattern of the glucose correlation value is the diabetes pattern C, the processor 12 extracts the voice data and the character data indicating advice 156 from the third data table 126.

Because diabetes and hyperlipidemia are suspected with this combination of the patterns, the advice is given to restrict diet.

As an example, the advice 156 is given as “Restrict your diet.”.

When the variation pattern of the neutral lipid correlation value is the hyperlipidemia pattern G and the variation pattern of the glucose correlation value is the hypoglycemia pattern D, the processor 12 extracts the voice data and the character data indicating advice 158 from the third data table 126.

Because hyperlipidemia is suspected with this combination of the patterns, the advice is given that physician's diagnosis is required.

As an example, the advice 158 is given as “Physician's diagnosis is recommended.”.

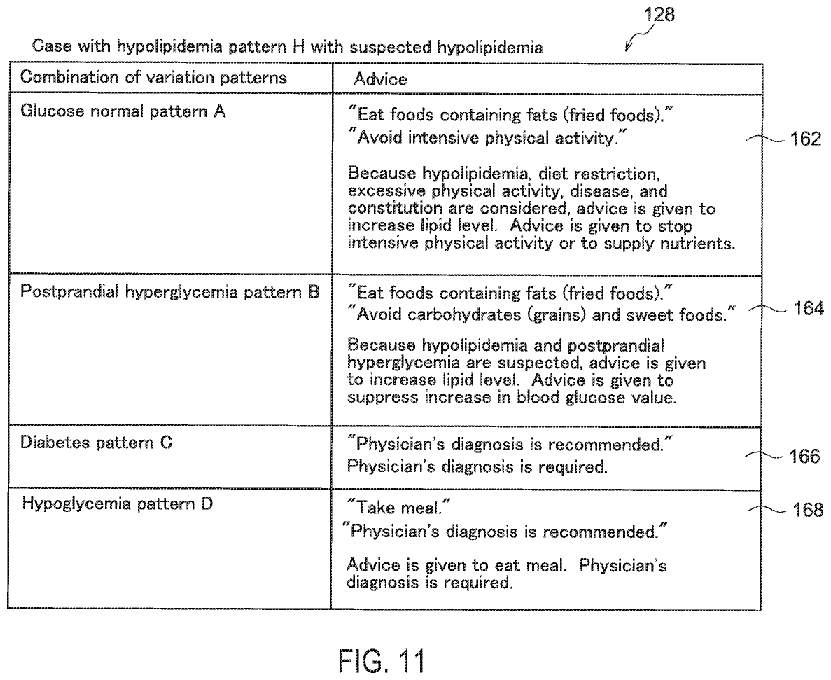

FIG. 11 is a diagram showing an example of the fourth data table 128 showing a relationship between the combinations of the variation patterns when hypolipidemia is suspected and the advices.

The fourth data table 128 stores the advices that correspond to the combinations of the hypolipidemia pattern H, in which the variation pattern of the neutral lipid correlation value indicates suspected hypolipidemia, with each of the patterns A, B, C, and D indicating the variation patterns of the glucose correlation value.

When the variation pattern of the neutral lipid correlation value is the hypolipidemia pattern H and the variation pattern of the glucose correlation value is the glucose normal pattern A, the processor 12 extracts the voice data and the character data indicating advice 162 from the fourth data table 128.

In a case with this combination of the patterns, the advice is given to increase the lipid level because hypolipidemia, diet restriction, the excessive physical activity, disease, or constitution are considered to be the cause. In addition, the advice is given to stop intensive physical activity and to supply nutrients.

As examples, the advice 162 is given as “Eat foods containing fats (fried foods).” and “Avoid intensive physical activity.”.

When the variation pattern of the neutral lipid correlation value is the hypolipidemia pattern H and the variation pattern of the glucose correlation value is the postprandial hyperglycemia pattern B, the processor 12 extracts the voice data and the character data indicating advice 164 from the fourth data table 128.

Because hypolipidemia and postprandial hyperglycemia are suspected with this combination of the patterns, the advice is given to increase the lipid level. In addition, the advice is given to suppress the increase in the glucose level (the blood glucose value).

As examples, the advice 164 is given as “Eat foods containing fats (fried foods).” and “Avoid carbohydrates (grains) and sweet foods.”.

When the variation pattern of the neutral lipid correlation value is the hypolipidemia pattern H and the variation pattern of the glucose correlation value is the diabetes pattern C, the processor 12 extracts the voice data and the character data indicating advice 166 from the fourth data table 128.

Because this combination of the patterns is a combination which is usually difficult to expect, the user is informed that physician's diagnosis is required.

As an example, the advice 156 is given as “Physician's diagnosis is recommended.”.

When the variation pattern of the neutral lipid correlation value is the hypolipidemia pattern H and the variation pattern of the glucose correlation value is the hypoglycemia pattern D, the processor 12 extracts the voice data and the character data indicating advice 168 from the fourth data table 128.

With this combination of the patterns, the advice is given to encourage the user to eat a meal. In addition, because anorexia is also suspected, the user is informed that physician's diagnosis is required.

As examples, the advice 168 is given as “Take a meal.” and “Physician's diagnosis is recommended.”.

The processor 12 then sets the advice by storing the advices extracted from the respective data tables 122 to 128 in an advice area reserved in the storage unit 30 so as to be readable (Step SC8), and the process returns to the advice generation process.

As shown in FIG. 4, in the advice generation process, the processor 12 executes exceedance advice process (Step SB4).

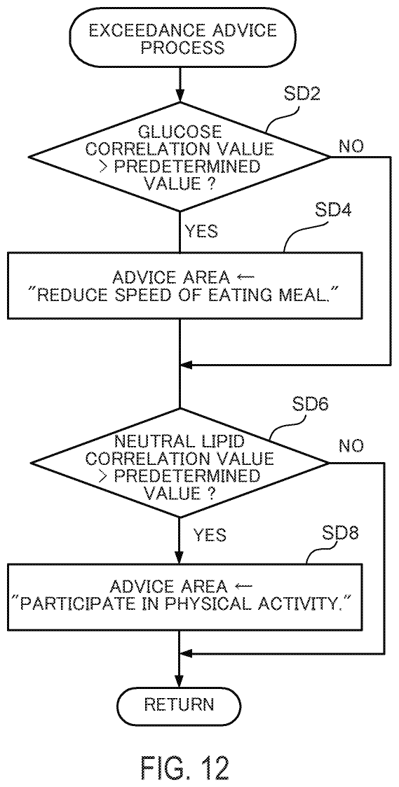

FIG. 12 is a flowchart showing an example of the exceedance advice process according to the first embodiment.

In the exceedance advice process, as shown in FIG. 12, the processor 12 determines whether or not the current glucose correlation value exceeds the predetermined value that is the first threshold value (Step SD2).

The first threshold value that is the predetermined value used in the determination in Step SD2 is, as an example, set to the same value as the first predetermined value 102 described above, and the first threshold value is set to 180 mg/dL as the glucose value.

In Step SD2, when it is determined that the current glucose correlation value is equal to or lower than the predetermined value that is the first threshold value, the process proceeds to Step SD6.

In addition, in Step SD2, when it is determined that the current glucose correlation value exceeds the predetermined value that is the first threshold value (see X in FIG. 6), the processor 12 generates the advice to reduce the speed of eating a meal (Step SD4). Specifically, the processor 12 stores the voice data and the character data indicating “Reduce the speed of eating a meal.” that is stored in advance in the storage unit 30 in the advice area reserved in the storage unit 30 so as to be readable.

The processor 12 then determines whether or not the current neutral lipid correlation value exceeds the predetermined value that is the second threshold value (Step SD6).

The second threshold value that is the predetermined value used in the determination in Step SD6 is, as an example, set to the same value as the third predetermined value 112 described above, and the second threshold value is set to 250 mg/dL as the neutral lipids value.

In Step SD6, when it is determined that the current neutral lipid correlation value is equal to or lower than the predetermined value that is the second threshold value (see Yin FIG. 7), the process returns to the advice generation process.

In addition, in Step SD6, when it is determined that the current neutral lipid correlation value exceeds the predetermined value that is the second threshold value, the processor 12 generates the advice to encourage the user to participate in the physical activity (Step SD8). Specifically, the processor 12 stores the voice data and the character data indicating “Participate in the physical activity.” that is stored in advance in the storage unit 30 in the advice area reserved in the storage unit 30 so as to be readable, and the process returns to the advice generation process.

Here, in this embodiment, the advice to reduce the speed of eating a meal is generated when the glucose correlation value exceeds the predetermined value that is the first threshold value. In addition, the advice to encourage the user to participate in the physical activity is generated when the neutral lipid correlation value exceeds the predetermined value that is the second threshold value. However, this embodiment is not limited thereto.

For example, the combination of the variation pattern of the glucose correlation value and the variation pattern of the neutral lipid correlation value may be added to the determination for generating the advice.

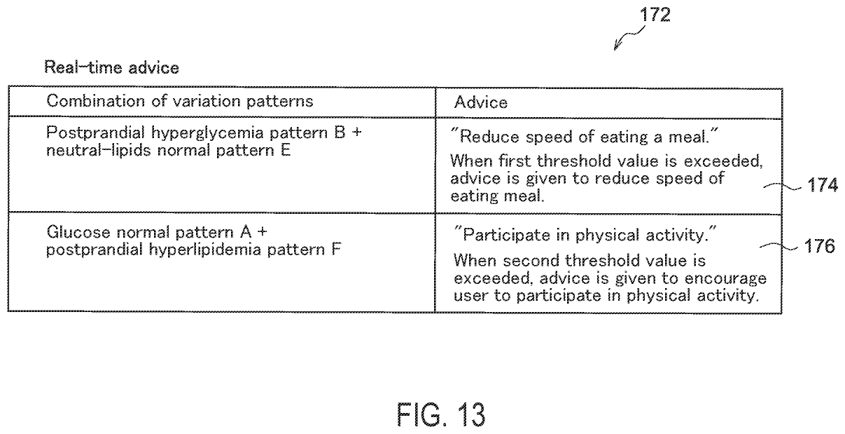

Specific example therefor will be described with reference to FIG. 13. FIG. 13 is a diagram showing an example of real-time advice. As shown in FIG. 13, a fifth data table 172 is stored in the storage unit 30.

When the advice is generated, the processor 12 determines whether or not the glucose correlation value exceeds the predetermined value that is the first threshold value when the variation pattern of the glucose correlation value is the postprandial hyperglycemia pattern B and the variation pattern of the neutral lipid correlation value is the neutral-lipids normal pattern E.

The first threshold value that is the predetermined value used in this determination is, as an example, set to the same value as the first predetermined value 102 described above, and the first threshold value is set to 180 mg/dL as the glucose value.

When the processor 12 determines that the glucose correlation value exceeds the predetermined value that is the first threshold value, the processor 12 extracts advice 174 from the fifth data table 172 and stores it in the advice area so as to be readable. The advice 174 is the voice data and the character data indicating “Reduce the speed of eating a meal.”.

In addition, the processor 12 determines whether or not the neutral lipid correlation value exceeds the predetermined value that is the second threshold value when the variation pattern of the glucose correlation value is the glucose normal pattern A and the variation pattern of the neutral lipid correlation value is the postprandial hyperlipidemia pattern F.

The second threshold value that is the predetermined value used in this determination is, as an example, set to the same value as the third predetermined value 112 described above, and the second threshold value is set to 250 mg/dL as the neutral lipids value.

When the processor 12 determines that the current neutral lipid correlation value exceeds the predetermined value that is the second threshold value, the processor 12 extracts advice 176 from the fifth data table 172 and stores it in the advice area so as to be readable, and the process returns to the advice generation process. The advice 176 is the voice data and the character data indicating “Participate in the physical activity.”.

Next, as shown in FIG. 4, in the advice generation process, the process returns to the blood component monitoring process.

As shown in FIG. 3, in the blood component monitoring process, the notification of the advice is performed (Step S10). Specifically, the processor 12 reads the voice data stored in the advice area in the storage unit 30 and outputs it from the notification unit 36 as a voice. In addition, the processor 12 reads the character data stored in the advice area in the storage unit 30 and outputs it from the display unit 34 as character display.

Here, when the stored amount of the glucose correlation values and the stored amount of the neutral lipid correlation values reach the predetermined value, and the variation pattern of the glucose correlation value and the variation pattern of the neutral lipid correlation value are determined, the advices corresponding to the respective combinations of the variation patterns are stored in the advice area. In this case, the advice is output from the notification unit 36 as the voice and output from the display unit 34 as the character display.

On the other hand, when the stored amount of the glucose correlation values and the stored amount of the neutral lipid correlation values do not reach the predetermined value, the variation pattern of the glucose correlation value and the variation pattern of the neutral lipid correlation value are not determined. In this case, the advices corresponding to the respective combinations of the variation patterns are not stored in the advice area.

However, when the glucose correlation value exceeds the predetermined value that is the first threshold value, the voice data and the character data indicating “Reduce the speed of eating a meal.” is stored in the advice area.

Therefore, at the time point when the glucose correlation value exceeds the predetermined value that is the first threshold value, the notification unit 36 outputs “Reduce the speed of eating a meal.” as the voice. In addition, the display unit 34 outputs “Reduce the speed of eating a meal.” as the character display.

In addition, when the neutral lipid correlation value exceeds the predetermined value that is the second threshold value, the voice data and the character data indicating “Participate in the physical activity.” are stored in the advice area.

Therefore, at the time point when the neutral lipid correlation value exceeds the predetermined value that is the second threshold value, the notification unit 36 outputs “Participate in the physical activity.” as the voice. In addition, the display unit 34 outputs “Participate in the physical activity.” as the character display.

As an example, on the basis of operated state of a finish button forming an input unit 23, it is determined whether or not the measurement is to be finished (Step S12). In Step S12, when it is determined that the finish button is not operated and the measurement is to be continued, the process is branched to Step S2 to continue the measurement. In addition, in Step S12, when it is determined that the finish button is operated and the measurement is to be finished, the blood component monitoring process is finished.

(Action and Effects)

Next, operational advantages of the blood component monitoring device 10 will be described.

The blood component monitoring device 10 in this embodiment is the blood component monitoring device 10 configured to monitor the blood component. The blood component monitoring device 10 includes the concentration acquisition unit 52 configured to acquire the glucose correlation value that correlates with the blood glucose concentration and the neutral lipid correlation value that correlates with the blood neutral lipid concentration. The blood component monitoring device 10 includes the information generation unit 54 configured to generate the information related to the blood status based on the variation patterns of the glucose correlation value and the neutral lipid correlation value caused by a meal.

According to this configuration, the information related to the blood status is generated based on the variation patterns of the glucose correlation value that correlates with the blood glucose concentration and the neutral lipid correlation value that correlates with the blood neutral lipid concentration.

Therefore, compared with a case in which the information is generated based on the blood sugar level only, it is possible to provide multifaceted information related to the blood status, in which not only sugars but also other components that are varied by a meal are taken into consideration.

For example, a case with high blood glucose concentration includes “a case in which both of the blood glucose concentration and the blood neutral lipid concentration are high” and “a case in which the blood glucose concentration is high, but the blood neutral lipid concentration is not high”. Thus, it is possible to provide a suitable content, in which the blood neutral lipid concentration is taken into consideration, as the advice to be given when the blood glucose concentration is high.

In addition, in this embodiment, the advice is generated as the information related to the blood status based on the variation patterns of the glucose correlation value that correlates with the blood glucose concentration and the neutral lipid correlation value that correlates with the blood neutral lipid concentration. Therefore, the advice corresponding to the one variation pattern can be finely differentiated according to the other variation pattern.

Therefore, compared with a case in which the advice is given on the basis of the blood sugar level only, it is possible to give finely categorized advices.

In addition, because the information related to the blood status is generated by using the variation patterns that change over time instead of the information of the blood component at a specific moment, it is possible to provide the information related to the blood status at appropriate timings corresponding to the variation.

For example, suppose that a threshold value is set to determine postprandial hyperglycemia, and a determination is performed whether a measurement result after a predetermined time is equal to or lower than the threshold value. In this case, if a predetermined value that is suitable for the determination of postprandial hyperglycemia is set as the threshold value, because the time required for the blood glucose value to fall varies from person to person, the blood glucose value (the glucose correlation value) would be determined as abnormal when the blood glucose value has not fallen to the lowest possible value.

Therefore, if the threshold value is set at a value higher than a predetermined value that is suitable for the determination of postprandial hyperglycemia, the threshold value is set rather high, and thus, erroneous detections are caused often.

In addition, the lipid level (the neutral lipid correlation value) varies over time depending on individual, as well as the meal content consumed by that individual. For example, the variation pattern of the neutral lipid correlation value that correlates with the lipid level varies widely, with the peak occurring in two hours or in four hours.

Therefore, it is difficult to predict the variation pattern of the neutral lipid correlation value (a waveform) using the neutral lipid correlation value at a certain point in time. Thus, if the information related to the blood status is generated by using the neutral lipid correlation value at a certain point in time, the information may be incorrect.

In contrast, in this embodiment, the information related to the blood status is generated by using the variation pattern of the neutral lipid correlation value. Therefore, prediction of the variation pattern of the neutral lipid correlation value is made unnecessary, and it becomes possible to improve an accuracy of the information related to the blood status.

Specifically described, even when the variation pattern of the glucose correlation value is the postprandial hyperglycemia pattern B, if the variation pattern of the neutral lipid correlation value is the neutral-lipids normal pattern E, there is no restriction for intake of lipids. In addition, even when the variation pattern of the glucose correlation value is the postprandial hyperglycemia pattern B, if the variation pattern of the neutral lipid correlation value is the hypolipidemia pattern H, there is no need to limit the intake of lipids.

Here, lipids are known to have a suppression effect on the glucose level.

Therefore, in a case with these combinations of the variation patterns, by encouraging the intake of lipids, it is possible to suppress a rapid increase in the glucose value (the blood glucose value).

Therefore, for the user with the postprandial hyperglycemia pattern B, it is possible to suppress the rapid increase in the glucose level that may occur after a meal.

In addition, in the blood component monitoring device 10 in this embodiment, the concentration acquisition unit 52 continuously acquires the glucose correlation value and the neutral lipid correlation value to acquire the temporal change of the glucose correlation value and the neutral lipid correlation value.

According to this configuration, compared with a case in which a measurement value measured before a meal, a measurement value measured after the meal, and their respective measurement times are used to acquire the variation pattern of the measurement values to give advices, it is possible to give suitable advices.

Furthermore, in the blood component monitoring device 10 in this embodiment, the information generation unit 54 generates the information related to the blood status on the basis of the combination of the variation patterns of the glucose correlation value and the neutral lipid correlation value.

According to this configuration, the information related to the blood status is generated on the basis of the combination of the variation patterns of the glucose correlation value and the neutral lipid correlation value. Therefore, compared with a case in which the advice serving as the information related to the blood status is generated on the basis of the combination of the measurement values measured after a meal, it is possible to give finely categorized suitable advices.

In addition, in the blood component monitoring device 10 in this embodiment, the information generation unit 54 generates the information related to the blood status when at least one of the glucose correlation value and the neutral lipid correlation value exceeds the predetermined value.

According to this configuration, it is possible to give the advice serving as the information related to the blood status when the glucose correlation value or the neutral lipid correlation value exceeds the predetermined value during a process in which the glucose correlation value or the neutral lipid correlation value is varied.

Furthermore, in the blood component monitoring device 10 in this embodiment, the information generation unit 54 generates the information related to the blood status for matters related to a meal when the glucose correlation value exceeds the first threshold value.

According to this configuration, it is possible to give the advice serving as the information related to the blood status related to a meal when the glucose correlation value exceeds the first threshold value during a process in which the glucose correlation value is varied.

In addition, in the blood component monitoring device 10 in this embodiment, the information generation unit 54 generates the information related to the blood status to reduce the speed of eating a meal when the glucose correlation value exceeds the first threshold value.

According to this configuration, it is possible to give the advice serving as the information related to the blood status for reducing the speed of eating a meal when the glucose correlation value exceeds the first threshold value during a process in which the glucose correlation value is varied. With this advice, as an example, the rapid increase in the glucose level can be suppressed.

Furthermore, in the blood component monitoring device 10 in this embodiment, the information generation unit 54 generates the advice to encourage the user to participate in the physical activity among the information related to the blood status when the neutral lipid correlation value exceeds the second threshold value.

According to this configuration, it is possible to give the advice to encourage the user to participate in the physical activity when the neutral lipid correlation value exceeds the second threshold value during a process in which the neutral lipid correlation value is varied. With this advice, as an example, the increase in the neutral lipid level can be suppressed.

In this embodiment, although a description has been given of a case in which the blood component monitoring device 10 form the respective units described above, the present invention is not limited thereto. For example, as shown in a second embodiment, the respective units may be formed of different devices.

### Second Embodiment

In the following, a blood component monitoring system according to the second embodiment will be described. Components that are the same as or similar to those in the first embodiment will be assigned the same reference numerals, and a description thereof shall be omitted. Description will only be given of components that are different from those in the first embodiment.

The blood component monitoring system according to the second embodiment is formed of a plurality of devices. The respective devices includes the concentration acquisition unit 52, the information generation unit 54, the combination advising unit 56, the exceedance advising unit 58, the eating speed advising unit 60, and the physical activity encouragement advising unit 62. The respective devices as a whole form the respective units.

As an example, the concentration acquisition unit 52 that acquires the glucose correlation value that correlates with the blood glucose concentration and the neutral lipid correlation value that correlates with the blood neutral lipid concentration is formed of a measurement device which can be worn by the user.

In addition, the information generation unit 54, the combination advising unit 56, the exceedance advising unit 58, the eating speed advising unit 60, and the physical activity encouragement advising unit 62 are formed of a terminal device used by the user.

The terminal device generates, as an example, the advice by obtaining the glucose correlation value that correlates with the blood glucose concentration and the neutral lipid correlation value that correlates with the blood neutral lipid concentration, which are acquired by the concentration acquisition unit 52, from the measurement device via wireless communication.

As described above, even with the blood component monitoring system formed of the measurement device and the terminal device, it is possible to achieve the operational advantages similar to those in the first embodiment.

Although the embodiments of the present invention have been described in the above, the above-mentioned embodiments merely illustrate a part of application examples of the present invention, and the technical scope of the present invention is not intended to be limited to the specific configurations of the above-described embodiments.

The present application claims priority to Japanese Patent Application No. 2021-058540, filed in the Japan Patent Office on Mar. 30, 2021. The contents of this application are incorporated herein by reference in their entirety.

### REFERENCE SIGNS LIST

10 blood component monitoring device
12 processor
14 measuring unit
30 storage unit
52 concentration acquisition unit
54 information generation unit
56 combination advising unit
58 exceedance advising unit
60 eating speed advising unit
62 physical activity encouragement advising unit
102 first predetermined value
104 second predetermined value
112 third predetermined value
114 fourth predetermined value
132, 134, 136, 138, 142, 144, 146, 148, 152, 154, 156, 158, 162, 164, 166, 168, 174, 176 advice
A glucose normal pattern
B postprandial hyperglycemia pattern
C diabetes pattern
D hypoglycemia pattern
E neutral-lipids normal pattern
F postprandial hyperlipidemia pattern
G hyperlipidemia pattern
H hypolipidemia pattern

What is claimed is:

1. A blood component monitoring device configured to monitor a blood component, the blood component monitoring device comprising:
a concentration acquisition circuit configured to acquire a glucose correlation value that correlates with a blood glucose concentration and a neutral lipid correlation value that correlates with a blood neutral lipid concentration; and
an information generation circuit configured to generate information related to blood status based on at least one of variation patterns of the glucose correlation value and the neutral lipid correlation value caused by a meal.

2. The blood component monitoring device according to claim 1, wherein
the concentration acquisition circuit is configured to acquire a temporal change of the glucose correlation value and the neutral lipid correlation value by continuously acquiring the glucose correlation value and the neutral lipid correlation value.

3. The blood component monitoring device according to claim 1, wherein
the information generation circuit is configured to generate the information related to blood status based on combination of variation patterns of the glucose correlation value and the neutral lipid correlation value.

4. The blood component monitoring device according to claim 2, wherein
the information generation circuit is configured to generate the information related to blood status based on combination of variation patterns of the glucose correlation value and the neutral lipid correlation value.

5. The blood component monitoring device according to claim 1, wherein
the information generation circuit is configured to generate the information related to blood status, when at least one of the glucose correlation value and the neutral lipid correlation value exceeds a predetermined value.

6. The blood component monitoring device according to claim 2, wherein
the information generation circuit is configured to generate the information related to blood status, when at least one of the glucose correlation value and the neutral lipid correlation value exceeds a predetermined value.

7. The blood component monitoring device according to claim 3, wherein
the information generation circuit is configured to generate the information related to blood status, when at least one of the glucose correlation value and the neutral lipid correlation value exceeds a predetermined value.

8. The blood component monitoring device according to claim 4, wherein
the information generation circuit is configured to generate the information related to blood status, when at least one of the glucose correlation value and the neutral lipid correlation value exceeds a predetermined value.

9. The blood component monitoring device according to claim 1, wherein
the information generation circuit is configured to generate the information related to blood status for matters related to a meal when the glucose correlation value exceeds a first threshold value.

10. The blood component monitoring device according to claim 2, wherein
the information generation circuit is configured to generate the information related to blood status for matters related to a meal when the glucose correlation value exceeds a first threshold value.

11. The blood component monitoring device according to claim 3, wherein
the information generation circuit is configured to generate the information related to blood status for matters related to a meal when the glucose correlation value exceeds a first threshold value.

12. The blood component monitoring device according to claim 4, wherein
the information generation circuit is configured to generate the information related to blood status for matters related to a meal when the glucose correlation value exceeds a first threshold value.

13. The blood component monitoring device according to claim 5, wherein
the information generation circuit is configured to generate the information related to blood status for matters related to a meal when the glucose correlation value exceeds a first threshold value.

14. The blood component monitoring device according to claim 9, wherein
the information generation circuit is configured to generate the information related to blood status for reducing a speed of eating a meal when the glucose correlation value exceeds the first threshold value.

15. The blood component monitoring device according to claim 1, wherein
the information generation circuit is configured to generate advice to encourage user to participate in physical activity among the information related to blood status when the neutral lipid correlation value exceeds a second threshold value.

16. The blood component monitoring device according to claim 2, wherein
the information generation circuit is configured to generate advice to encourage user to participate in physical activity among the information related to blood status when the neutral lipid correlation value exceeds a second threshold value.

17. The blood component monitoring device according to claim 3, wherein
the information generation circuit is configured generate advice to encourage user to participate in physical activity among the information related to blood status when the neutral lipid correlation value exceeds a second threshold value.

18. The blood component monitoring device according to claim 4, wherein
the information generation circuit is configured to generate advice to encourage user to participate in physical activity among the information related to blood status when the neutral lipid correlation value exceeds a second threshold value.

19. A non-transitory computer-readable recording medium including a blood component monitoring program configured to cause a processor to monitor a blood component to execute:
a concentration acquisition procedure of acquiring a glucose correlation value that correlates with a blood glucose concentration and a neutral lipid correlation value that correlates with a blood neutral lipid concentration; and
an information generation procedure of generating information related to blood status based on at least one of variation patterns of the glucose correlation value and the neutral lipid correlation value caused by a meal.

20. A blood component monitoring system having a plurality of devices for monitoring a blood component, the blood component monitoring system comprising:
a concentration acquisition circuit configured to acquire a glucose correlation value that correlates with a blood glucose concentration and a neutral lipid correlation value that correlates with a blood neutral lipid concentration; and
an information generation circuit configured to generate information related to blood status based on at least one of variation patterns of the glucose correlation value and the neutral lipid correlation value caused by a meal.
